(19) United States
(12) Patent Application Publication
(10) Pub. No.: US 2022/0160619 A1
(43) Pub. Date: May 26, 2022

(54) Effervescent Drug Formulations

(71) Applicant: Complete Medical Solutions, LLC, Ponte Vedra Beach, FL (US)

(72) Inventor: Charles Richardson, Ponte Vedra Beach, FL (US)

(73) Assignee: Complete Medical Solutions, LLC, Ponte Vedra Beach, FL (US)

(21) Appl. No.: 17/104,238

(22) Filed: Nov. 25, 2020

(51) Int. Cl. A61K 9/46; A61K 31/155; A61K 31/714; A61K 47/12; A61K 31/519

(52) U.S. Cl. CPC A61K 9/0007; A61K 31/155; A61K 31/519; A61K 47/12; A61K 31/714

(57) ABSTRACT

The disclosure relates to an effervescent therapeutic composition for delivering multiple compounds to a patient in a single administration. In some embodiments, the effervescent therapeutic composition includes an alkaline effervescing compound, one or more compressible binders, a pharmaceutically acceptable salt of metformin, a pharmaceutically acceptable cobalamin, and an acid compound or an acid salt. In other embodiments, the effervescent therapeutic composition may include a folate or folic acid and metformin. In yet other embodiments, the effervescent therapeutic composition may include a folate or folic acid, cobalamin, and metformin.

### FIELD OF INVENTION

The present invention relates generally to the preparation, formulation, and administration of certain compounds to a patient as a medical treatment. More particularly, the present disclosure relates to specific formulations of effervescent treatments for targeting a plurality of mechanisms and pathologies in a disease state.

### BACKGROUND OF THE INVENTION

Many diseases and illnesses are a combination of various mechanisms, reactions, and symptoms that are identified by a single title. For example, diabetes mellitus is a combination of various metabolic disorders that result in increased levels of blood glucose. Those increased levels of blood glucose then result in other pathologies. It is common that a single disease or illness requires a combination of medications to be administered to the patient during the course of the treatment. In cases of chronic diseases, the constant administration of multiple medications results in low patient compliance and therefore ineffective treatment.

Some of the reasons given by non-compliant patients include difficulty SW lowing large pills, unpleasant aftertaste, multiple administrations of various pills in a day, complicated doses or equipment for administration while travelling, bulky equipment for administration with irregular routine, etc. These problems are very common with elderly patients, who are often taking multiple treatments throughout the day including prescription drugs, vitamins, and supplements. Elderly patients may also suffer from dysphagia, which hinders their ability to take pills, especially large pills.

With specific reference to diabetic patients, certain studies have found multiple physiological abnormalities in patients that require multiple therapeutic compounds to be administered. In one study, it was found that cobalamin malabsorption was present in 30% of diabetic patients taking long-term metformin therapy in addition to dietary management. In the same study it was found that the patients experiencing cobalamin malabsorption had significantly lower hemoglobin levels (and significantly higher serum folic acid levels) than those with normal cobalamin absorption. Likewise, it was found that the cessation of metformin therapy resulted in reversion of cobalamin absorption to normal levels in most patients.

What is needed then are improvements to administration of treatments for pathologies requiring multiple administrations or difficult administrations, specifically in situations where it is difficult for certain patients to ingest the types and quantities therapeutic compounds.

### BRIEF SUMMARY

This Brief Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

One aspect of the disclosure is an effervescent therapeutic composition, including an alkaline effervescing compound, one or more compressible binders, a pharmaceutically acceptable salt of metformin, a pharmaceutically acceptable cobalamin, and an acid compound or an acid salt.

Another aspect of the disclosure is an effervescent therapeutic composition, including an alkaline effervescing compound, one or more compressible binders, a pharmaceutically acceptable salt of metformin, a pharmaceutically acceptable folate, and an acid compound or an acid salt.

A further aspect of the disclosure is an effervescent therapeutic composition, including an alkaline effervescing compound, one or more compressible binders, a pharmaceutically acceptable salt of metformin, a pharmaceutically acceptable folate, a pharmaceutically acceptable cobalamin and an acid compound or an acid salt.

Another aspect of the disclosure is a method for preparing an effervescent therapeutic composition, including the steps of mixing and drying the various compounds disclosed, including an alkaline effervescing compound, one or more compressible binders, a pharmaceutically acceptable salt of metformin, a pharmaceutically acceptable folate, a pharmaceutically acceptable cobalamin, and an acid compound or an acid salt.

Numerous other objects, advantages and features of the present disclosure will be readily apparent to those of skill in the art upon a review of the following drawings and description of a preferred embodiment.

### BRIEF DESCRIPTION OF THE DRAWINGS

FIG. 1 is a perspective view of an exemplary embodiment of an effervescent therapeutic tablet prior to insertion into a liquid.

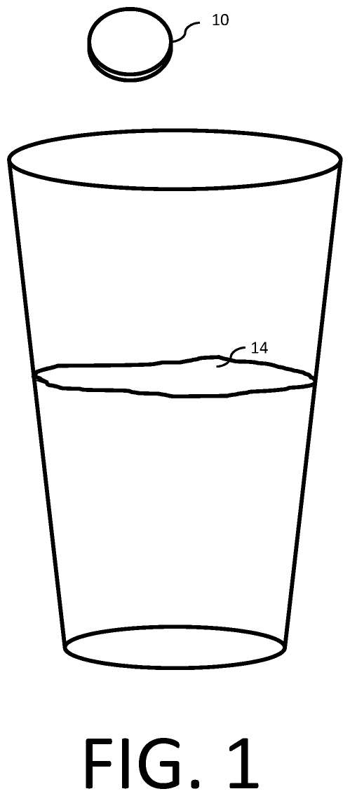

FIG. 2 is a perspective view of an exemplary embodiment of an effervescent therapeutic tablet after to insertion into a liquid, wherein the tablet is releasing pharmaceutically acceptable compounds into solution.

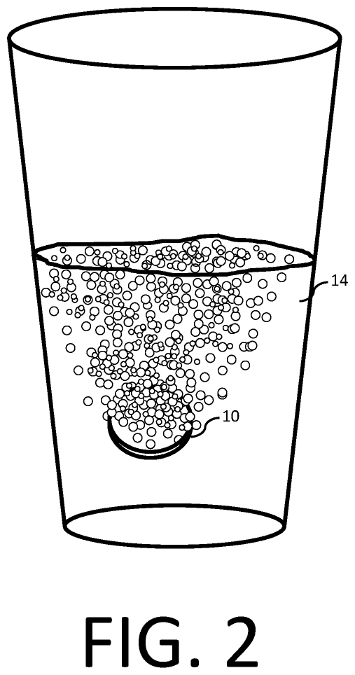

FIG. 3 is a perspective view of an exemplary embodiment of an effervescent therapeutic powder being poured into a liquid prior to contact.

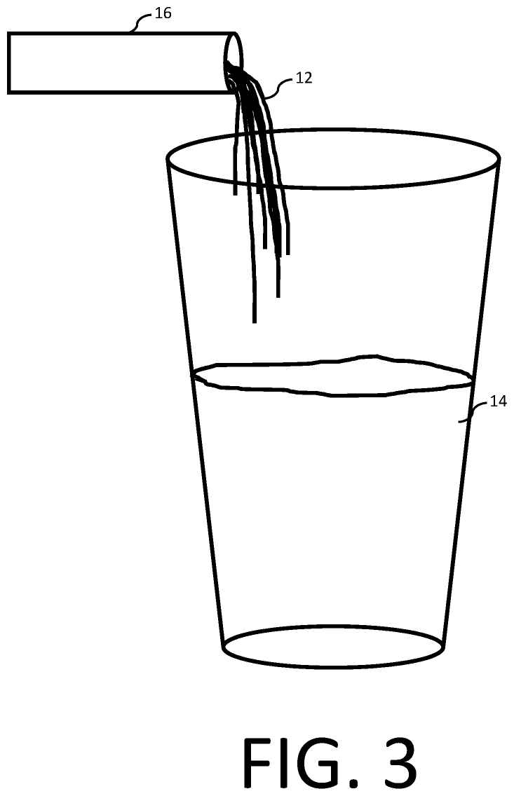

FIG. 4 is a perspective view of an exemplary embodiment of an effervescent therapeutic powder being poured into a liquid and during contact, wherein the powder is releasing pharmaceutically acceptable compounds into solution.

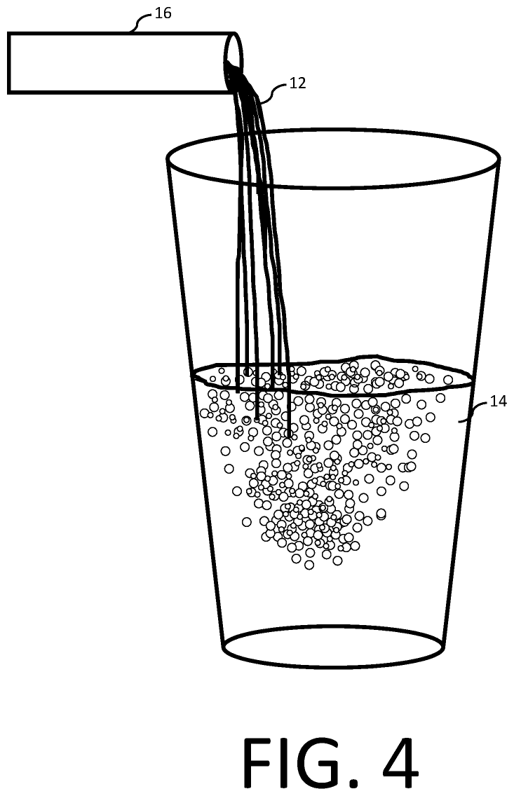

### DETAILED DESCRIPTION

While the making and using of various embodiments of the present invention are discussed in detail below, it should be appreciated that the present invention provides many applicable inventive concepts that are embodied in a wide variety of specific contexts. The specific embodiments discussed herein are merely illustrative of specific ways to make and use the invention and do not delimit the scope of the invention. Those of ordinary skill in the art will recognize numerous equivalents to the specific apparatus and methods described herein. Such equivalents are considered to be within the scope of this invention and are covered by the claims.

In the drawings, not all reference numbers are included in each drawing, for the sake of clarity. In addition, positional terms such as “upper,” “lower,” “side,” “top,” “bottom,” etc. refer to the apparatus when in the orientation shown in the drawing. A person of skill in the art will recognize that the apparatus can assume different orientations when in use.

The present disclosure relates, in part, to the difficulty of administering treatments to patients. This is especially true for treatments requiring multiple oral administrations of multiple compounds for a single disease to a patient who has difficulty swallowing. Although specific diseases and disease states will be discussed with specificity, the specific examples are not to be construed as limiting on the scope of the disclosure to a single formulation or administration.

The use of effervescing therapeutic compounds includes numerous benefits to the patient and for patient compliance. Effervescent therapeutic compounds are generally effective at providing an even distribution of the compound to patient in comparison to a conventional tablet. Traditional tablets rely upon dissolution in the stomach for the compounds to be in a state to be absorbed by the patient. Often, the tablet will be only partially dissolved which limits the compound that will be absorbed by the patient. Furthermore, partially dissolved tablets can lead to irritation of the patient's stomach and along the digestive tract. In contrast, effervescent tablets 10 dissolve completely and evenly, which prevents localized areas of higher and lower concentrations of the compounds during administration. In contrast, effervescent therapeutic compounds may be dissolved entirely prior to administration thus evenly distributing the therapeutic compounds in the liquid and to the patient. This can be extremely beneficial to elderly patients as, after the age of 40, the digestive system becomes less efficient in performing its function in breaking down and absorbing the material passing through it.

Likewise, effervescing therapeutic compounds are easier for patients who have difficulty swallowing, especially large pills. Many therapeutic compounds are delivered orally and require the patient to swallow a solid tablet, and often the solid tablet is large, making it difficult for some to swallow. Elderly or young patients often experience dysphagia, which makes it extremely difficult to ingest the therapeutic compounds. In some cases, the pills can be broken into smaller sizes and crushed, which can make it easier for the patient to ingest. However, in some cases this is not appropriate as the encased compound may be prematurely activated, or the solid tablet is meant to be slow release. Thus, the patient may inadvertently disrupt the intended delivery mechanism and, in some cases, endanger themselves by altering the tablet.

In contrast, because effervescent therapeutic compounds are easy to ingest when dissolved properly in water, the patient does not alter the therapeutic compound and is able to ingest even in cases of dysphagia. Furthermore, the effervescent therapeutic compounds are easy to carry, administer, and often have fewer complaints because of taste and aftertaste.

Effervescent therapeutic compounds can also deliver large doses of ingredients

in a single administration. Furthermore, because the effervescent therapeutic compounds are dissolved in a liquid 14, the patient's fluid intake is increased which can be beneficial in many circumstances when a patient has a reduced appetite. Often, effervescent compounds are easily palatable as the ingredients used often include citric acid or other palatable compounds. The effervescent compound may also be dissolved in other liquids 14 such as fruit juices. The effervescent compounds may also react in such a way to form a natural buffer when in solution, thus reducing damage or irritation to the digestive tract.

Likewise the use of effervescent therapeutic compounds may be preferred in those embodiments in which certain compounds are difficult to digest or are disruptive to the stomach (such as producing gas or resulting in constipation). Furthermore, because effervescent compounds must be protected from moisture until they are prepared for administration, some therapeutic compounds may ideally be delivered in an effervescent format when those compounds are sensitive to light, oxygen, or moisture. Often effervescent therapeutic compounds are packaged in unit including aluminum, which is intended to block out moisture light, and oxygen.

Thus, the use of effervescent therapeutic compounds can be beneficial for increasing patient compliance, delivering consistent and controlled therapeutic compounds to the patient, and delivering large quantities and multiple compounds in one administration.

As a first example, diabetes mellitus is a complex, chronic illness requiring continuous medical care with multifactorial risk reduction strategies beyond glycemic control. Diabetes mellitus has been linked to a variety of molecular interactions and biological pathways related to the production of insulin, cell receptors and glucose uptake, mitochondrial function, and more. Each of these deficient interactions and pathways can lead to further complications. The following symptoms and complications are often associated with diabetes mellitus: increased thirst, increased hunger (especially after eating) dry mouth, frequent urination, unexplained weight loss, weak or tired feeling, blurred vision, numbness or tingling in the hands or feet, slow-healing sores or cuts, dry itchy skin, frequent yeast or urinary tract infections, sweating, pounding heart, pale skin, anxiety, confusion, poor coordination, difficulty focusing, numbness in mouth and tongue, and passing out.

Significant evidence exists that supports a range of interventions to improve diabetes outcomes. Disclosed herein is a compound relating to an effervescent composition of metformin, including an alkaline effervescing compound, at least one compressible binder, one or more pharmaceutically acceptable salts of metformin, one or more secondary treatment compounds, and an acid compound or an acid salt.

For example, a secondary treatment compound may include cobalamin. Cobalamin is essential to hemopoetic, neuro-cognitive, and cardiovascular function. Specifically, cobalamin has been found to promote various biological functions in humans, including, but not limited to, fatty acid and amino acid metabolism, the synthesis of myelin, the maturation of red blood cells, DNA synthesis, etc. Patients being treated for diabetes mellitus with metformin have demonstrated a susceptibility to cobalamin deficiencies. It has also been found that metformin-associated cobalamin deficiencies increase due to age, dosage of metformin, and duration of use of metformin. In some embodiments, cobalamin may refer to Vitamin B12, either naturally occurring or synthetic variants thereof.

Another example of a secondary treatment compound includes folate or folic acid. Folate promotes the formation of red blood cells. Folate is also a significant contributor to building and repairing skin cells in the human body. Folate is also responsible for replacing various other types of old cells with new cells. The cells found in the small intestine lining are produced using folate. Folate is also a coenzyme, which effectively works in association with enzymes to perform the essential functions of the body, for example, DNA synthesis. Folate is responsible for improving hemoglobin levels. Hemoglobin is an essential component in oxygen transfer to cells and organ systems. Thus, folate can increase energy levels and increase metabolic efficiency. In some embodiments, folate or folic acid may refer to Vitamin B9, either naturally occurring or synthetic variants thereof.

By producing effervescent therapeutic compounds with multiple therapeutic agents, the patient is able to receive, in a single administration, doses of multiple compounds that would otherwise have to be ingested in separate administrations. Likewise, in some embodiments, it may be advantageous to administer the compounds together as the compounds may be synergistic. For example, a compound may be administered together with a coenzyme involved in the metabolization of the compound. In another example, a first compound may be administered with a second compound, wherein deficiencies of the second compound are linked to patients taking the first compound. Another example may include where a first compound and a second compound promote absorption of the other when administered together. Although these examples provide examples of scenarios in which it may be beneficial to administer two therapeutic compounds in one administration, one of skill in the art will recognize that various other reasons for administering two compounds in one administration is desirable.

Now discussing a second example of disease that may be treated with the disclosed therapeutic compounds, Polycystic Ovary Syndrome, also known as Stein-Leventhal Syndrome, is a heterogeneous disorder of chronic anovulation and hyperandrogenism believed to result from a hormonal imbalance created by a combination of increased androgens and/or insulin. Often associated with obesity and insulin resistance, Polycystic Ovary Syndrome symptoms include menstrual dysfunction, acne, hirsutism, obesity, infertility, insulin resistance, and polycystic ovaries by ultrasonography. Patients are at increased risk for type 2 diabetes, metabolic syndrome, infertility, high cholesterol, high blood pressure and heart disease. EX404 is an age appropriate formulation of an existing molecule for adolescent girls with Polycystic Ovary Syndrome.

Now turning to a discussion of the specific formulations and methods for preparing effervescing therapeutic compounds, as previously discussed, may include an alkaline effervescing compound, one or more compressible binders, one or more pharmaceutically acceptable salts of metformin, one or more secondary treatment compounds, and an acid compound or an acid salt.

The acid component of the formulation may include citric acid, tartaric acid, malic acid, fumaric acid, and adipic acid. In one embodiment, citric acid is used for its relatively pleasant taste, which contributes to an overall taste of the effervescent compound and increased patient compliance. Likewise, salts of inorganic acids may be utilized in the formulation. Any combination of each of these acids and/or acid salts may be utilized in various relative ratios to achieve desired results such as reactivity, taste, stability, etc.

The basic component of the formulation may include sodium bicarbonate, sodium carbonate, and sodium sesquicarbonate. Alternatively, basic component of the formulation may include potassium bicarbonate, potassium carbonate, potassium sesquicarbonate, and potassium glycine carbonate. In some embodiments, the use of potassium-based components may be advantageous in decreasing sodium consumption of the patient, which has been linked to several adverse results from excessive sodium consumption. Furthermore, any combination of each of these basic components may be utilized in various relative ratios to achieve desired results such as reactivity, taste, stability, etc.

In one embodiment, the acid or acid salts present in the described formulation is greater than the basic or carbonate source. Alternatively, the acids and bases may be present in equal amounts. By increasing the ratio of the acids compared to the salts, the carbonate source is more fully expended during the reaction and dissolution when an effervescent tablet 10 or powder 12 is mixed with a liquid 14. Likewise, the acid is often the more palatable flavor and is therefore preferred from a practical standpoint to have excess acid after the dissolution has occurred. However, the molar ratio of the acids and bases may be varied due to the nature of each acid and base. For example, when potassium carbonate is used, two moles of a weak organic acid such as citric acid will be used with three moles of potassium carbonate due to the nature of the reactants.

The effervescent therapeutic compound in some embodiments will also include a buffering compound. The buffering compound and the amounts in each specific formulation will vary depending on the acids, bases, and other compounds present. However, one of skill in the art will recognize that any buffering compound that is effective to maintain a pH of 4-7 or 5-6 and is safe for consumption may be used in combination with the disclosed effervescent therapeutic compound.

One of skill in the art will recognize acceptable compressible binders for use in adhering the compounds disclosed herein into a tablet 10 may include, in some embodiments, granules. Because of the nature of the effervescing therapeutic compound, it is generally preferable to use a dry binder. For example, starch binders may be used in the disclosed embodiments.

In one embodiment, the effervescing therapeutic compound may include metformin or a pharmaceutically acceptable salt of metformin. As previously discussed, metformin may be used in the treatment of diabetes mellitus or Polycystic Ovary Syndrome.

In some embodiments, a secondary treatment compound may include cobalamin. In other embodiments, a secondary treatment compound may include folate or folic acid. Each of these secondary treatment compounds is discussed in more detail above.

The effervescent therapeutic compound may either be presented as an effervescent tablet 10 or a powder 12. When preparing a tablet 10 with the effervescent therapeutic compound, the effervescent tablet 10 may include a range of total weight. This may allow the patient to be provided various amounts of the therapeutic compounds ranging from low-dose treatments to high-dose treatments. For example, the effervescent tablet 10 may be 500 to 5,000 mg. A 5,000 mg dose would represent a high-dose treatment, whereas a 500 mg dose would represent, in some embodiments, a low-dose treatment. In other embodiments, the effervescent tablet 10 may include a 1,000 to a 2,500 mg dose. In other embodiments, the effervescent tablet 10 may include a 3,000 to a 4,500 mg dose. In each of the embodiments, the various component parts of the effervescing tablet 10 may vary to represent pharmaceutically acceptable levels of each compound.

Likewise, effervescent powders 12 may be prepared in varying dosages as discussed above with reference to effervescent tablets 10. As portions of the preparation of the effervescent tablets 10 and powders 12 are similar, those portions will be discussed together. Powders 12 may be stored in a variety of containers, including but not limited to packets 16, pouches, jars, etc.

Because effervescent tablets 10 and powders 12 react in the presence of water, the preparation requires a low humidity environment when preparing the effervescent therapeutic compounds. Thus, dehumidifiers, silica beads, or other methods known to one of skill in the art from controlling moisture content may be implemented in certain steps of the preparation of the disclosed compounds.

In some embodiments, a first step includes a first preparation. The first preparation includes the acidic portions, which in some embodiments may be citric or tartaric acid, sugar alcohols, water, and, in some instances, flavor additives. The first preparation is then granulated and dried. The drying process is an important factor as discussed above in order to prevent premature effervescing when the acid and base are combined.

The first preparation, after drying, is combined with the other compounds discussed above, which have likewise been properly dried to prevent premature effervescing. These component parts may be mixed over a period of time in dehumidified conditions. The formulations may also include certain compounds suitable for a delayed release of the therapeutic compounds, which allows the compounds to be slowly absorbed by the patient.

Specific effervescent therapeutic compounds may include a combination of metformin or metformin salts with folate or folic acid. The combination of metformin and folic acid may be beneficial for diabetic patients that are suffering from folate deficiencies. Thus, this specific combination may provide for increased blood glucose level regulation and metabolism. Likewise, the combination may improve certain conditions prevalent in diabetic and folate deficient patients such as increased cellular oxygen levels and cellular repair.

A second specific effervescent therapeutic compound may include a combination of metformin or metformin salts with cobalamin. The combination of metformin and cobalamin may be beneficial for diabetic patients that are suffering from cobalamin deficiencies. Because metformin has been linked to cobalamin deficiencies in patients, it is beneficial for patients to receive supplements of cobalamin during periods of metformin use. Likewise, because it has been shown that after patients have discontinued use of metformin that cobalamin deficiencies ceased to persist in most patients, it is beneficial to the patient to cease cobalamin supplementation when metformin is no longer being administered to the patient. Thus, a combination of metformin and cobalamin may be administered in a single effervescent therapeutic compound to increase blood glucose level regulation, metabolism, neural function, cellular reproduction, and cellular oxygen levels.

In a third embodiment, an effervescent therapeutic compound may include a combination of metformin salts, cobalamin, and folate or folic acid. As discussed above with reference to the specific combinations disclosed, it may be beneficial for diabetic patients to receive the standard metformin treatment with supplements of both cobalamin and folate. This may be advantageous to patients suffering from diabetes, cobalamin deficiency and folate deficiency. When all three are administered in an effervescent therapeutic compound, the results may include an increase in blood glucose level regulation, metabolism, neural function, cellular reproduction, cellular repair, and cellular oxygen levels.

As patients with Polycystic Ovary Syndrome are likewise often treated with metformin, the disclosed combinations may likewise be applicable for providing an effervescent therapeutic compound for treating Polycystic Ovary Syndrome. Although the specific combinations have been disclosed with specific reference to specific pathologies, this disclosure is not intended to be limited to the specific referenced diseases. One of skill in the art will recognize that the various compounds disclosed may be used for a variety of diseases, including but not limited to, congestive heart failure, breast cancer, prostate cancer, reducing risk of stroke and dementia, and aging.

Thus, although there have been described particular embodiments of the present invention of a new and useful EFFERVESCENT DRUG FORMULATIONS, it is not intended that such references be construed as limitations upon the scope of this invention.

What is claimed is:

1. An effervescent therapeutic composition, comprising:
an alkaline effervescing compound;
at least one compressible binder;
a pharmaceutically acceptable salt of metformin;
a pharmaceutically acceptable cobalamin; and
an acid compound or an acid salt.

2. The effervescent therapeutic composition of claim 1, wherein at least one of the acid compound and the acid salt compound include citric acid.

3. An effervescent therapeutic composition, comprising:
an alkaline effervescing compound;
at least one compressible binder;
a pharmaceutically acceptable salt of metformin;
a pharmaceutically acceptable folate; and
an acid compound or an acid salt.

4. The effervescent therapeutic composition of claim 3, wherein at least one of the acid compound and the acid salt compound include citric acid.

5. An effervescent therapeutic composition, comprising:
an alkaline effervescing compound;
at least one compressible binder;
a pharmaceutically acceptable salt of metformin;
a pharmaceutically acceptable folate;
a pharmaceutically acceptable cobalamin; and
an acid compound or an acid salt.

6. The effervescent therapeutic composition of claim 5, wherein at least one of the acid compound and the acid salt compound include citric acid.
